SEQUENCE LISTING (16 sequences)

SEQ ID NO: 1 - DNA, 17 bp, Artificial Sequence
  Beta-Actin primer
cctggcacccagcacaa

SEQ ID NO: 2 - DNA, 20 bp, Artificial Sequence
  Beta-Actin primer
gccgatccacacggagtact

SEQ ID NO: 3 - DNA, 26 bp, Artificial Sequence
  Beta-Actin primer
atcaagatcattgctcctcctgagcg

SEQ ID NO: 4 - DNA, 23 bp, Artificial Sequence
  CD4 primer
ggaaatcagggctccttcttaac

SEQ ID NO: 5 - DNA, 22 bp, Artificial Sequence
  CD4 primer
gtcccaaaggcttcttcttgag

SEQ ID NO: 6 - DNA, 24 bp, Artificial Sequence
  CD4 primer
ccatccaagctgaatgatcgcgct

SEQ ID NO: 7 - DNA, 18 bp, Artificial Sequence
  CD25 primer
cgatgacccgccagagat

SEQ ID NO: 8 - DNA, 24 bp, Artificial Sequence
  CD25 primer
cattcacagttcaacatggttcct

SEQ ID NO: 9 - DNA, 24 bp, Artificial Sequence
  CD25 primer
ccacacgccacattcaaagccatg

SEQ ID NO: 10 - DNA, 20 bp, Artificial Sequence
  CTLA-4 primer
cgccagctttgtgtgtgagt

SEQ ID NO: 11 - DNA, 18 bp, Artificial Sequence
  CTLA-4 primer
cctgccgaagcactgtca

SEQ ID NO: 12 - DNA, 24 bp, Artificial Sequence
  CTLA-4 primer
tgcatctccaggcaaagccactga

SEQ ID NO: 13 - DNA, 18 bp, Artificial Sequence
  IL-10 primer
cggcgctgtcatcgattt

SEQ ID NO: 14 - DNA, 26 bp, Artificial Sequence
  IL-10 primer
tggagcttattaaaggcattcttcac

SEQ ID NO: 15 - DNA, 26 bp, Artificial Sequence
  IL-10 primer
tccacggccttgctcttgttttcaca

SEQ ID NO: 16 - DNA, 20 bp, Artificial Sequence
  CpG oligodeoxynucleotide
tccatgacgttcctgacgtt

(19) United States
(12) Patent Application Publication
(10) Pub. No.: US 2003/0049696 A1
(43) Pub. Date: Mar. 13, 2003

(54) Regulatory T cells and uses thereof

(76) Inventors: Anne M. Norment, Seattle, WA (US); Thaddeus C. George, Seattle, WA (US)

Correspondence Address: IMMUNEX CORPORATION, LAW DEPARTMENT, 51 UNIVERSITY STREET, SEATTLE, WA, 98101

(21) Appl. No.: 10/164,776

(22) Filed: Jun. 6, 2002

Related U.S. Application Data
(60) Provisional application No. 60/296,586, filed on Jun. 7, 2001.
(60) Provisional application No. 60/303,564, filed on Jul. 6, 2001.

(51) Int. Cl. G01N 33/567; G01N 33/574; C12N 5/08

(52) U.S. Cl. 435/7.21; 435/7.23; 435/372

(57) ABSTRACT

Regulatory T cell subpopulation (Treg) are isolated for a human host by selection for cells expressing CD4 and CD25. The Treg cells are further characterized by expression of CTLA-4, CCR6, and CD30. In addition, the Treg cells are CD62L^{hi}, CD45RB^{lo}, CD45RO^{hi}, CD45RA⁻. The Treg cells of the invention reflect the immunologic status of the donor, in terms of the number, location and T cell antigen receptor specificity of the Treg cells. This information is used in diagnostic assays relating to immunologic disorders, e.g. cancer related immunosuppression; autoimmune disorders; atopic states, etc. The isolated Treg cells are useful in transplantation, for experimental evaluation, and as a source of subset and cell specific products, including mRNA species useful in identifying genes specifically expressed in these cells, and as targets for the discovery of factors or molecules that can affect them. Culture assays and systems of interest include the interactions of Treg cells with immature and mature dendritic cells, interactions with T cell subsets, responsiveness to antigen specific and non-specific stimulus, and the like.

### BACKGROUND OF THE INVENTION

A healthy immune system reacts against harmful pathogens while remaining specifically tolerant to autologous tissues. Failure of such self tolerance can result in autoimmune disease, while a failure to respond appropriately can lead to infection, and may result in the unchecked growth of tumor cells. Putting immunotherapy into practice is a highly desired goal in the treatment of such human diseases. The basis for immunotherapy is the manipulation of the immune response, particularly the responses of T cells. T cells possess complex and subtle systems for controlling their interactions, utilizing numerous receptors and soluble factors for the process.

For most autoimmune diseases, atopic states and undesired immune responses, no effective diagnostic blood tests or therapeutic agents exist. For example, current therapeutic strategies are often based on chemically induced immunosuppression, which can result in undesirable side effects on the kidney and other organs. However, the search for naturally occurring immunosuppressive molecules and cell types has been long, with some notable red herrings.

For many years it was believed that antigen-specific suppressor T cells existed that were restricted by the “IJ” region of the major histocompatibility complex, and were CD8⁺. More recent experiments in animal models have suggested that there is a different subset of T cells with suppressive regulatory activity. In the mouse, the subset has been characterized as expressing both CD4 and CD25. CD4 is a marker both for some thymocyte populations and for helper T cells, which has a role in the formation of complexes between the T cell antigen receptor and MHC antigens. CD25 is a component of the receptor for IL-2, and can be a marker for activated T cells. Interestingly, in the mouse, T cells that are CD4⁺ CD25+ can be either a regulatory T cell subset (Treg) that contains autoimmune-preventive activity, or activated T helper cells that contain substantial autoreactive potential.

The mouse cells that have suppressive regulatory activity are thymically derived, express a polyclonal TCR repertoire, and make up 5-10% of spleen and lymph node CD4⁺ T cells. In addition to expression of CD4 and CD25, they are predominantly CD62L^{hi} and CD69⁻, the latter distinguishing them from activated CD4⁺ T cells. These regulatory cells express markers characteristic of memory T cells, for example they are CD45RB^{low}, CD44^{hi}, perhaps reflecting stimulation in vivo by self antigen.

The mouse Treg cells are also functionally distinct from normal CD4⁺ T helper (Th) cells. Unlike conventional CD4⁺ T cells, Treg cells fail to proliferate or to secrete cytokines in vitro in response to antigen presenting cells and antigenic stimulus. However they are not completely anergic, and can respond to some combinations of factors. An interesting feature of regulatory T cells is their ability to inhibit the proliferative response of normal T helper cells in vitro, as well as their secretion of IL-2. Interestingly, several stimuli which appear to break Treg anergy also inhibit Treg cell function in a co-culture assay.

Little is known about the mechanism by which murine Treg cells inhibit T helper cell proliferation in vitro, much less their ability to modulate immune activation in vivo. Cell to cell contact seems to be required. And while stimulation of the T cell antigen receptor appears to be necessary for induction of Treg activity in co-culture experiments, experiments with T cells from TCR transgenic mice have indicated that it is not necessary for the regulatory and helper T cells to have the same TCR specificity to get an inhibition of proliferation. Engagement of CTLA-4 also appears to be required for suppression. Mouse Treg cells express IL-10 and TGF-β, and although mAb to IL-10 and TGF-β do not block Treg function in vitro, in vivo experiments have indicated the importance of IL10.

Little more is known about the possible human counterparts to these mouse cells, and of the molecular mechanisms that control Treg expansion, activation, or effector function. Understanding how Treg cells are activated and how they regulate the immune response will be important to understanding the regulation of autoimmunity.

Relevant Literature

A number of studies have been directed at regulatory T cell populations in mouse and rat models. For example, Sakaguchi (2000) Cell 101:455-458 reviews the role of regulatory T cells in the control of self-tolerance. Thornton and Shevach (2000) J. Immunol. 164:183-190 discuss the antigenic specificity of mouse T regulatory cells; and Kuniyashi et al. (2000) Int. Immunol. 12:1145-1155 discuss the expression of CD25 on these cells. International patent application WO00/42856 suggests that alpha melanocyte stimulating hormone induces T regulatory cells. A relationship between Tumor immunity and T regulatory cells is suggested by Shimizu et al. (1999) J. Immunol. 163:5211-5218. The expression of CTLA-4 on mouse regulatory T cells is shown by Takahashi et al. (2000) J.E.M. 192:303-309. Jordan et al. (2001) Nat. Immunol. 2:301 demonstrates selection of Treg cells in murine thymus on thymic stromal cells, which selection required high avidity interactions between their TCR and self-peptide MHC. Thorton and Shevach (1998) J.E.M. 188:287-96 describe in vitro Treg assays.

Additional relevant literature on human CD4+CD25+ regulatory T cells may be found in Jonuleit et al. (2001) J.E.M. 193:1285-94; Levings et al. (2001) J.E.M. 193:1295-1301; Dieckmann et al. (2001) J.E.M. 193:1303-1310; and Yamagiwa et al. (2001) J. Immunol. 166:7282-89, Stephens et al. (2001) Eur. J. Immunol. 31:1247-1254; and Taams et al. (2001) Eur. J. Immunol. 31:1122-1131.

Stephens and Mason (2000) J. Immunol. 165:3105-3110 discuss the expression of CD25 in rat thymocytes. Interactions between dendritic cells and regulatory T cells is discussed in Dhodapkar et al. (2001) J.E.M. 193:233-238; Roncarolo et al. (2001) J.E.M. 193:F5-F9; and Jonuleit et al. (2000) J.E.M. 192:1213-1222.

Tr1 or TH3-like regulatory T cell clones from human peripheral blood CD4⁺ T cells are described by Groux et al. 1997 Nature 389:737; Kitani et al. (2000) J. Immunol. 165:691-702; and Fukaura et al. (1996) J. Clin. Invest. 98:70. A commentary on human regulatory T cells may be found in Waldman and Cobbold (2001) Immunity 166:3008-3018.

### SUMMARY OF THE INVENTION

A substantially enriched human regulatory T cell subpopulation (Treg) is provided, which is characterized by the ability of the cells to specifically suppress immune responses, particularly T cell mediated immune responses. Methods are provided for the isolation and culture of this regulatory T cell from natural sources, e.g. peripheral blood. The cell enrichment methods may employ reagents that specifically recognize CD25 and CD4. Optionally CD69 or CD45RA are used in a negative selection. Subsets of the Treg population may be isolated using reagents that are specific for one or more of the markers including CCR6, CD30, CTLA-4, CD62L, CD45RB, and CD45RO.

The cells of the invention are generally derived from an in vivo source, and therefore reflect the immunologic status of the donor, in terms of the number, location and T cell antigen receptor specificity of the Treg cells. This information is used in diagnostic assays relating to immunologic disorders, e.g. cancer related immunosuppression; autoimmune disorders; atopic states, etc.

The Treg cells of the invention are useful in transplantation for the transfer of immunosuppression, for experimental evaluation, and as a source of subset and cell specific products, including mRNA species useful in identifying genes specifically expressed in these cells, and as targets for the discovery of factors or molecules that can affect them. In vitro systems are provided for the growth and analysis of Treg cells. Culture assays and systems of interest include the interactions of Treg cells with immature and mature dendritic cells, interactions with T cell subsets, responsiveness to antigen specific and non-specific stimulus, and the like.

### BRIEF DESCRIPTION OF THE DRAWINGS

FIG. 1 shows sorted human Treg and Th cells.

FIGS. 2A and 2B shows that sorted human Treg express CD30 and CCR6, respectively.

FIG. 3 illustrates that human Treg are anergic and inhibit the proliferation of human CD4 T helper cells in vitro.

FIG. 4 shows that the Treg cell numbers are increased by Flt3-L treatment and shows the percentage of murine Treg and Th cells in spleen, lymph node and blood.

FIG. 5 shows increased expression of CTLA 4 and IL-10 in Treg cells.

FIG. 6 shows that Treg cells stimulated with antigen dose and activated APC retain the ability to suppress Th proliferation, but with sufficient stimulation, Th cells can escape Treg-mediated suppression of proliferation.

### DESCRIPTION OF THE SPECIFIC EMBODIMENTS

Methods of enrichment and substantially enriched human cell populations are provided. Human regulatory T cell subpopulation (Treg) are characterized as a CD4⁺CD25⁺CD69⁻ population, which population can inhibit immune responses, including, for example, the proliferation of human CD4⁺CD25⁻ T cells. Subsets of the Treg cells are further characterized by expression of CCR6, and CD30. In addition, the Treg cells are CD62L^{hi}, CD45RB^{lo}, CD45RO^{hi}, CD45RA⁻.

Methods are provided for the isolation and culture of this regulatory T cell from natural sources, e.g. peripheral blood, apheresis blood product, leukopheresis blood product, etc. The cell enrichment methods may employ specific binding reagents that recognize CD25, CD4, optionally and/or CD45RA, may optionally utilize reagents specific for one or more of the markers including CD69, CCR6, CD30, CTLA-4, CD62L, CD45Rb, and CD45RO.

In a preferred embodiment, the Treg cells are isolated from a human donor, which donor may be immunologically normal, or may suffer from an immunological disorder relating to immunosuppression. Disorders of interest include immunosuppressive conditions, e.g. cancer, certain parasitic infections, e.g. trypanosomiasis; AIDS; and the like. Conditions of interest also include conditions where there is a loss of immunosuppression, e.g. autoimmune and other pro-inflammatory diseases. Analysis of donor Treg may include both absolute and relative numbers, localization with sites of the body, expression levels of specific co-stimulatory molecules such as members of the CD28 family, e.g. CD28, CTLA-4, ICOS, PD-1, etc.; expression of TNFR family proteins, e.g. OX40, CD30, 4-1 BB, etc.; expression of chemokine receptors; the specific profile of T cell antigen receptors expressed by the Treg cells; and the like. This information is used in diagnostic assays, for therapeutic intervention, etc.

The Treg cells of the invention are useful in transplantation for the transfer of immunosuppression, for experimental evaluation, and as a source of subset and cell specific products, including mRNA species useful in identifying genes specifically expressed in these cells, and as targets for the discovery of factors or molecules that can affect them. In vitro systems are provided for the growth and analysis of Treg cells. Culture assays and systems of interest include the interactions of Treg cells with immature and mature dendritic cells, interactions with T cell subsets, responsiveness to antigen specific and non-specific stimulus, and the like. The interactions of Treg cells or Treg progenitors with poor antigen presenting cells, which may include human monocytes, B cells, macrophages, etc. are also of interest for culture systems and assays, as the interactions with such cells may stimulate Treg effector function, support Treg expansion or stimulate differentiation of T cells into the Treg pathway.

### Separation of TREG Cells

The Treg cells of the present invention can be enriched on the basis of expression of cell surface markers. The cells are positively selected for expression of CD4 and CD25, and can be negatively selected for the absence of CD45RA. Optionally, other markers can be used to further separate subpopulations of the Treg cells, including CD69, CCR6, CD30, CTLA-4, CD62L, CD45RB, and CD45RO. The methods can include further enrichment or purification procedures or steps for cell isolation by positive selection for other cell specific markers.

In vivo sources of cell populations useful as a source of cells include, but are not limited to peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, and the like. The donor is preferably human, and can be fetal, neonatal, child, adult, and may be normal, diseased, or susceptible to a disease of interest.

The subject Treg cells are separated from a complex mixture of cells by techniques that enrich for cells having the characteristics of being CD4⁺CD25⁺, and optionally CD45RA⁻. For isolation of cells from tissue, an appropriate solution may be used for dispersion or suspension. Such a solution will generally be a balanced salt solution, e.g. normal saline, PBS, Hank=s balanced salt solution, etc., conveniently supplemented with fetal calf serum, BSA, normal goat serum, or other naturally occurring factors, in conjunction with an acceptable buffer at low concentration, generally from 5-25 mM. Convenient buffers include HEPES, phosphate buffers, lactate buffers, etc.

Separation of the subject cell population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, LDS). Any technique may be employed which is not unduly detrimental to the viability of the selected cells.

The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody, i.e. using a saturating amount of antibody. The appropriate concentration can also be determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The staining intensity of cells can be monitored by flow cytometry, where lasers detect the quantitative levels of fluorochrome (which is proportional to the amount of cell surface antigen bound by the antibodies). Flow cytometry, or FACS, can also be used to separate cell populations based on the intensity of antibody staining, as well as other parameters such as cell size and light scatter. Although the absolute level of staining may differ with a particular fluorochrome and antibody preparation, the data can be normalized to a control.

The labeled cells are then separated as to the expression of CD4 and CD25. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove=s medium, etc., frequently supplemented with fetal calf serum.

Compositions highly enriched for human Treg activity are achieved in this manner. The subject population will be at or about 70% or more of the cell composition, and usually at or about 90% or more of the cell composition, and may be as much as about 95% or more of the cell population. The enriched cell population may be used immediately. Cells can also be frozen, although it is preferable to freeze cells prior to the separation procedure, or may be frozen at liquid nitrogen temperatures and stored for long periods of time, being thawed and capable of being reused. The cells will usually be stored in DMSO and/or FCS, in combination with medium, glucose, etc. Once thawed, the cells may be expanded by use of growth factors, antigen, stimulation, dendritic cells, etc. for proliferation and differentiation.

### In vitro Models and Uses

The present methods are useful in the development of in vitro models and assays for human Treg cell function and are also useful in experimentation on gene expression and cellular interactions. The Treg cells serve as a valuable source of novel regulatory factors and pharmaceuticals. The enriched cell population may be grown in vitro under various culture conditions. Culture medium may be liquid or semi-solid, e.g. containing agar, methylcellulose, etc. The cell population may be conveniently suspended in an appropriate nutrient medium, such as Iscove's modified Dulbecco's medium, or RPMI-1640, normally supplemented with fetal calf serum (about 5-10%), L-glutamine, and antibiotics, e.g. penicillin and streptomycin.

The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Specific growth factors that may be used in culturing the subject cells include the interleukins, e.g. IL-1, IL-2, IL-3, IL-4, IL-5, IL-6, IL-7, IL-8, IL-8, IL-10, IL-11, IL-12, IL-13, IL-14, IL-15, IL-16, IL-17, IL-18, etc.; antigens, e.g. peptide antigens, protein antigens such as alloantigens, preferably in combination with antigen presenting cells; lectins, e.g. Con A; α-CD3; LPS; etc. The culture may also contain antibodies, or specific ligands (in the form of purified ligand, Fc fusion proteins, or other recombinant tagged forms like leucine zipper forms) for cell surface receptors that may stimulate or inhibit Treg activity. For example, mAb or ligands that bind TNFR or other co-stimulatory molecules on Treg and could stimulate and increase Treg activity, override Treg activity (and induce proliferation), or that stimulate apoptosis of Treg can be included. The specific culture conditions are typically chosen to achieve a particular purpose, i.e. maintenance of Treg cell activity, etc.

The subject co-cultured cells may be used in a variety of ways. For example, the culture medium may be isolated at various stages and the components analyzed. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions that contain Treg effector molecules that inhibit the proliferation of helper T cells, or contain Treg molecules that may act in an autocrine fashion to maintain the regulatory state of Treg cells.

The Treg cells may be used in conjunction with immature or mature dendritic cells, as well as other antigen presenting cells, e.g. monocytes, B cells, macrophages, etc. in a culture system in the isolation and evaluation of factors associated with the initiation of Treg activity. Thus, the cells may be used in assays to determine the activity of media, such as conditioned media, evaluate fluids for factor activity, or the like. In addition, an antigen presenting cell free culture system may be devised for the expansion of Treg cells using soluble growth factors and/or mAb or ligands for Treg cell surface receptors.

The subject cells may be used for suppression of immune function in a recipient. Allogeneic or autologous cells may be used for isolation, modification in vitro, and subsequent transplantation. The cells may be administered in any physiologically acceptable medium, normally intravascularly, including intravenous, although they may also be introduced into other convenient sites.

Genes may be introduced into the cells prior to culture or transplantation for a variety of purposes, e.g. prevent or reduce susceptibility to infection, replace genes having a loss of function mutation, increase Treg potency to inhibit Th cells, make Treg home to specific regions, etc. Alternatively, vectors are introduced that express antisense mRNA or ribozymes, thereby blocking expression of an undesired gene. Other methods of gene therapy are the introduction of drug resistance genes to enable normal cells to have an advantage and be subject to selective pressure, for example the multiple drug resistance gene (MDR), or anti-apoptosis genes, such as bcl-2. Various techniques known in the art may be used to transfect the target cells, e.g. electroporation, calcium precipitated DNA, fusion, transfection, lipofection and the like. The particular manner in which the DNA is introduced is not critical to the practice of the invention.

Many vectors useful for transferring exogenous genes into mammalian cells are available. The vectors may be episomal, e.g. plasmids, virus derived vectors such cytomegalovirus, adenovirus, etc., or may be integrated into the target cell genome, through homologous recombination or random integration, e.g. retrovirus (including lentivirus) derived vectors such MMLV, HIV-1, ALV, etc.

### Analysis of TREG Cells

The subject cells are useful for in vitro assays and screening to detect factors that are active on Treg cells. Assays of interest also include co-culture assays to study alterations in the ability of Treg to inhibit proliferation of normal T cells, including CD4 T as well as CD8 T. Interaction with dendritic cells and other antigen presenting cells are also of interest. A wide variety of assays may be used for this purpose, including immunoassays for protein binding; determination of cell growth, differentiation and functional activity; production of hormones; and the like.

Analysis of the interaction between dendritic cell types and Treg cells, particularly with respect to differences with Th1 and Th2 T cells is of particular interest. Treg cell effector function may be preferentially elicited in vivo by interaction with a specific DC subset or DCs in a particular activation state, where self antigens that are presented by immature/tolerogenic DCs may serve to maintain peripheral tolerance by inducing Treg function. Other less potent APC such as B cells or monocytes may also participate in this process. Cross-talk between Treg cells and DC or other APC may also go in the converse direction, with Treg cells affecting the expansion, activation or co-stimulatory capacity of particular DC or other APC types.

Of particular interest is the examination of gene expression in human Treg cells, in the absence or presence of dendritic cells types and growth/regulatory factors of interest. The expressed set of genes may be compared with a variety of cells of interest, e.g. Th1 cells, memory T cells, Th2 cells, CTL, thymocytes, etc., as known in the art. For example, one could perform experiments to determine the genes that are regulated during response to differing antigenic stimulus. Human and mouse cells may also be compared.

In one screening method, the test sample is assayed at the protein level. Diagnosis can be accomplished using any of a number of methods to determine the absence or presence or altered amounts of a differentially expressed polypeptide in the test sample. For example, detection can utilize staining of cells or histological sections (e.g. from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

Any suitable qualitative or quantitative methods known in the art for detecting specific mRNAs can also be used. mRNA can be detected, for example, by hybridization to a microarray, in situ hybridization in tissue sections, by reverse transcriptase-PCR, or in Northern blots containing poly A⁺ mRNA. One of skill in the art can readily use these methods to determine differences in the size or amount of mRNA transcripts between two samples. For example, the level of particular mRNAs in Treg cells is compared with the expression of the mRNAs in a reference sample, e.g. naive T helper cells, memory T helper cells, etc.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample can be analyzed using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

### In vivo Diagnosis and Therapy

The analysis of Treg cells in a patient is useful for determining specific markers of immunosuppression, including specific antigenic specificities that may be absent or present, the location within the body of Treg cells, and the number of Treg cells, both in absolute numbers and in relation to Th1 and/or Th2 cells. CD4⁺CD25⁺ Treg cells have the capacity to suppress autoimmune responses in several in vivo murine models, while depletion of Treg cells leads to organ specific auto-immune diseases. Just as enhancing or mimicking Treg cell function may represent an important avenue to treat autoimmune disease, blocking Treg cell function may augment anti-tumor responses in cancer patients. CD4⁺ T cell responses in cancer patients are markedly down-modulated; inhibiting the function of Treg cells may provide an important strategy to stimulate anti-tumor immunity.

Formats for patient sampling include time courses that follow the progression of disease, comparisons of different patients at similar disease stages, e.g. early onset, acute stages, recover stages, etc.; tracking a patient during the course of response to therapy, including drug therapy, vaccination and the like. An important consideration is using studies of Treg to give information about the effects of particular immunomodulating agents. For example, the absolute number of Treg and the ratio of Treg/T helper is increased in Flt3-L treated animals, so evaluating the effects of immunomodulating agents on Treg can be important for analyzing the efficacy of the agents in treating cancer or autoimmune disease. The effect of Flt3-L may be used to enhance the number of Treg cells in an animal, by administering an effective dose of Flt3-L, which increases the total number of Treg cells and/or mobilizes Treg cells. Data from animals, e.g. mouse, rat, rabbit, monkey, etc. may be compiled and analyzed in order to provide databases detailing the course of disease, antigens involved in diseases, etc.

Biological samples from which patient antibodies may be collected include blood and derivatives therefrom, e.g. leukopheresis product, apheresis product, etc. Other sources of samples are body fluids such as synovial fluid, lymph, cerebrospinal fluid, bronchial aspirates, and may further include saliva, milk, urine, and the like. Cells may be collected from blood, tissues such as spleen, thymus, lymph nodes, fetal liver, tissues at the site of autoimmune lesions, e.g. pancreas, joints, cerebrospinal fluid, etc., tumors, blood from patients with metastatic disease, etc. The Treg cells may be analyzed intact, or lysates may be prepared for analysis.

Methods for quantitation of cells and detection of antigenic specificity are known in the art, and may include pre-labeling the sample directly or indirectly; adding a second stage antibody that binds to the antibodies or to an indirect label, e.g. labeled goat anti-human serum, rat anti-mouse, and the like. For example, see U.S. Pat. No. 5,635,363.

Generally assays will include various negative and positive controls, as known in the art. These may include positive controls of “spiked” samples with known autoantibodies, patients with known disease, and the like. Negative controls include samples from normal patients, animal serum, and the like.

Various methods are used to determine the antigenic specificity profile from a patient sample. The comparison of a binding pattern obtained from a patient sample and a binding pattern obtained from a control, or reference, sample is accomplished by the use of suitable deduction protocols, Al systems, statistical comparisons, pattern recognition algorithms, etc. Typically a data matrix is generated, where each point of the data matrix corresponds to a readout from specific epitope. The information from reference patterns can be used in analytical methods to determine relative abundance, changes over time, etc.

Tumors of interest for treatment include carcinomas, e.g. colon, duodenal, prostate, breast, ovarian, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g. neuroblastoma, gliomas, etc.; hematological malignancies, e.g. chronic myologenous leukemia, childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Autoimmune disease of interest include asthma, systemic lupus erthymatosus, rheumatoid arthritis, type I diabetes, multiple sclerosis, Crohn's disease, ulcerative colitis, psoriasis, myasthenia gravis, etc.

### Modulation of Regulatory Behavior

Murine Treg cells have been found to exist in both a regulatory and proliferative state, which state can reflect the Treg cell's response to quantitative and qualitative properties of antigenic or other stimulus. In response to low levels of antigenic stimulation, for example at antigen concentrations of less than about 5 nM, the Treg cells do not proliferate and are capable of suppressing T cell proliferation and responses in a non-antigen specific manner. Thus, agents that modulate human Treg activity, for example by delivering either strong or weak antigenic stimulation, are of interest. These agents may include, without limitation, antibodies or ligands to cell-surface receptors that deliver co-stimulatory signals to Treg, as well as agents modulating the antigen presenting capacity of APC to Treg.

For example, signaling through co-stimulatory molecules, such as CD30 or 4-1BB may affect the signaling that causes human Treg cells to enter the proliferating, or regulatory state. Ligand binding to such co-stimulatory molecules may cause Treg cells to proliferate, and thereby inhibit their effector cell activity, possibly leading to apoptosis of Treg. Antibodies that bind to CD30/4-1BB or CD30ligand/4-1BB ligand treatment can be used to modify the regulatory behavior of Treg. Alternatively, stimulation through CD30 or 4-1BB may enhance Treg effector function by stimulating up regulation of immunosuppressive factors.

### Modulation of TREG Trafficking

Methods are provided to specifically modulate the trafficking of regulatory T cells. Regulatory T cells express high levels of the chemokine receptor CCR6. It may be noted that immature dendritic cells, which have been implicated in the initial differentiation Treg cells, also express CCR6. In response to chemokine receptor agonists, leukocytes are triggered to undergo integrin-dependent arrest at a target site. This arrest acts to localize the cells at the target site. In some embodiments of the invention, this trigger is manipulated to modulate the adhesion of these regulatory T cells to endothelial cells. The methods of the invention may also modulate the chemotaxis of these T cells, which may also control their trafficking and interactions in sites of inflammation. The role of chemokines in leukocyte trafficking is reviewed by Baggiolini (1998) Nature 392:565-8, in which it is suggested that migration responses in the complicated trafficking of lymphocytes of different types and degrees of activation will be mediated by chemokines. The use of small molecules to block chemokines is reviewed by Baggiolini and Moser (1997) J. Exp. Med. 186:1189-1191.

In the subject methods, compounds that modulate the triggering activity of CCR6 are administered systemically or locally to alter the trafficking behavior of the regulatory T cells. Trafficking, or homing, is used herein to refer to the biological activities and pathways that control the localization of leukocytes in a mammalian host. Such trafficking may be associated with disease, e.g. inflammation, allergic reactions, etc., or may be part of normal biological homeostasis.

Local administration that provides for a prolonged localized concentration, which may utilize sustained release implants or other topical formulation, is of particular interest. In one embodiment of the invention the trigger modulating compound is an agonist of CCR6, which acts to enhance the triggering effect. In an alternative embodiment, the trigger modulating compound blocks CCR6 activity. In vivo uses of the method are of interest for therapeutic and investigational purposes. In vitro uses are of interest for drug screening, determination of physiological pathways, and the like. The subject methods also provide for targeting cells from blood to skin and other systemic sites of inflammation by expressing CCR6 on the cells to be targeted.

CCR6 modulating agents are molecules that specifically act as an agonist to enhance CCR6 biological activity; or that act as antagonists that block CCR6 biological activity, for example the interaction between CCR6 and its ligands. Often such agents interact with the extracellular binding domain or transmembrane domain of CCR6 protein, and may activate the molecule through the ligand binding site, block the ligand binding site, conformationally alter the receptor, etc. Usually the binding affinity of the blocking agent will be at least about 100 μM. Preferably the blocking agent will be substantially unreactive with related molecules to CCR6, such as CCR1, CCR2, CCR3, CCR4, etc., particularly CCR7; and other members of the seven transmembrane domain superfamily. Blocking agents do not activate CCR6 triggering of adhesion. Agonists may activate the triggering activity, enhance chemotaxis activity, or enhance the triggering activity of other ligands.

CCR6 modulating agents are peptides, small organic molecules, peptidomimetics, antibodies, or the like. Antibodies are an exemplary modulating agent. Antibodies may be polyclonal or monoclonal; intact or truncated, e.g. F(ab′)₂, Fab, Fv; xenogeneic, allogeneic, syngeneic, or modified forms thereof, e.g. humanized, chimeric, etc. Naturally occurring ligands of CCR6 include LARC, and MIP-3 alpha. MIP-3 alpha is normally associated with inflamed epithelium, a site of antigen entry known to be infiltrated by immature DC.

In many cases, the modulating agent will be an oligopeptide, e.g. antibody or fragment thereof, etc., but other molecules that provide relatively high specificity and affinity may also be employed. Combinatorial libraries provide compounds other than oligopeptides that have the necessary binding characteristics. Generally, the affinity will be at least about 10⁻⁶, more usually about 10⁻⁸ M, i.e. binding affinities normally observed with specific monoclonal antibodies.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### EXAMPLE 1

### Human Treg

The following describes methods for isolating a population of CD4⁺CD25⁺ Treg cells in human from human peripheral blood mononuclear cells (PBMC). For example, using negative bead depletion of Ficoll-Hypaque purified human PBMC leukopheresis preps, CD4⁺ T cells at ˜95% purity can be obtained. These cells can then be stained with anti-CD4 and anti-CD25 mAb and sorted on the flow cytometer. In a typical prep, 10⁹ PBMC yield 2-5×10⁸ CD4⁺ T cells, which are sorted to give 1-3×10⁶ CD4⁺CD25⁺ cells (>95% pure) and an excess (>5×10⁷) of CD4⁺CD25⁻ Th responder cells. Flow cytometry of sorted CD4⁺CD25⁺ cells showed that they are CD69⁻, CD62L^{hi}, CD45RB^{low}, CD45RO^{hi}. Gated human CD4⁺CD25⁺ cells are also CD45RA⁻.

In order to determine proliferation characteristics of human CD4⁺CD25⁺ cells, in vitro proliferation assays were performed. Sorted CD4⁺CD25⁺ cells were compared to CD4⁺CD25⁻ cells for their ability to proliferate in culture in response to autologous monocytes in the presence of ConA (1 μg/ml). Human CD4⁺CD25⁺ cells were anergic (failed to proliferate) while normal CD4⁺ Th cells proliferated well. Importantly, in a dose dependent manner human CD4⁺CD25⁺ cells inhibited the proliferation of CD4⁺CD25⁻ cells, with >90% inhibition when mixed in a 1:1 ratio. The foregoing demonstrates that human PBMC contain a population of regulatory T cells analogous to murine Tregs.

To further characterize the human CD4⁺CD25⁺ regulatory T cells, gene expression patterns were evaluated. Notably, human CD4⁺CD25⁺ T cells expressed 20-fold or 10-fold more mRNA for CTLA-4 and IL-10, respectively, which molecules have been shown to be important in the function of murine Treg cells. Human Treg, unlike CD4⁺CD25⁻ cells, expressed cell-surface CD30, but not detectable OX-40 or 4-1BB. CD30 and 4-1BB mRNA is increased >20-fold in human Treg compared to Th. Thus human peripheral blood derived Treg constituitively express CD30.

Materials and Methods

Antibodies and flow cytometry. The following anti-human mAb were purchased from BD Pharmingen (San Diego, Calif.): Cy-Chrome-anti-CD4, APC-anti-CD8α (RPA-T8), FITC-anti-CD25 (clone M-A251), APC-anti-CD25 (M-A251), PE-anti-CD21,PE-anti-CD45RB (MT4), PE-anti-CD45RO (UCHL1), PE-anti-CD45RA (HI100), PE-anti-CD69, PE-anti-CD62L (L-selectin), PE-anti-CD134 (OX40), PE-anti-CDw137 (4-1 BB), PE-anti-CCR6, PE-anti-HLA-DR, PE-anti-CD80, Biotin-anti-CD30, APC-anti-CD56 (B159). In some cases, the folowwing antibodies were used for staining: PE-anti-CD25 and Biotin-anti-CD25 (both 143-13, Biosource International, Camarillo, Calif.), and PE-anti-CD25 (M-A251, BD Pharmingen). Staining with biotinylated Ab reagents utilized FITC-, PE- or APC-Streptavidin (BD Pharmingen). For CCR7 detection, staining utilized anti-CCR7 (unconjugated IgM mAb), followed by biotin-anti-mouse-IgM and PE-Streptavidin according to the manufacturer's instructions (BD Pharmingen). Biotin-anti-CD30L (clone M82 Reference: Richard Armitage RJ. CD153. In Leucocyte Typing VI ed. T. Kishimoto et al., Garland Publishing, Inc., New York, p98-100, 1997.) was generated in house following affinity purification on protein A Sepharose. FITC-anti-CD14 was from BD Pharmingen (M5E2) and subsequently Immunotech (Marseille, France). PE-anti-CD3 was from Immunotech. Staining was performed by standard methods on ice in PBS containing 1% normal rabbit serum and 2% normal goat serum (Sigma, St. Louis, Mo.), in the presence of the anti-Fc receptor mAb (anti-CDw32, clone IV.3). Staining was performed on Ficoll purified PBMC, purified CD4 T cells, or sorted T cells (see isolation procedures below). Samples were washed and fixed in 1% paraformaldehyde in PBS prior to collection on a FACSCalibur machine, and subsequent analysis using CellQuest software (Becton Dickinson, San Jose, Calif.).

Freezing and Thawing of PBMC For the best mode of cell freezing, PBMC were resuspended in 75% FBS, 10% DMSO, and 3% glucose in RPMI 1640 with 2-5×10⁸ cells per cryovial in Cryo Freezing containers (NALGENE, Nalge Nunc International, Milwaukee, Wis.) at −80° C., and subsequently transferred to liquid nitrogen tanks. Other freezing protocols include 20% FBS, 10% DMSO in culture media, or 50% FBS, 12% DMSO in RPMI 1640. For thawing cells, PBMC are quickly thawed at 37° C. and then added dropwise at room temperature to 4 ml FBS in a 50 ml conical tube. Thawed PBMC are then washed twice in media and then counted and assessed for viability using a haemocytometer and trypan blue exclusion.

Isolation of PBMC, CD4 T cells and Sorted CD4+CD25+ Regulatory T cells or CD4+CD25− Helper T cells. Heparinized venous blood was drawn from healthy volunteers. Leukopheresis product was obtained by standard procedures from healthy donors. In some experiments leukopheresis product was also obtained from a commercial vendor (AllCells, Berkeley, Calif.) following overnight shipping in autologous plasma. For blood samples, PBMC were isolated from blood or leukopheresis product, by Ficoll density gradient centrifugation using using lsolymph (Gallard-Schlesinger Industries Inc., Carle Place, N.Y.) according to standard methods. Blood and in some cases leukopheresis product were treated with Red blood cell lysis buffer (Sigma) and then washed twice with PBS. CD4 T cells were then isolated by negative magnetic bead selection using the CD4⁺ T Cell Isolation Kit followed by separation using AutoMACS columns according to the manufacturer's instructions (Miltenyi Biotec, Bergisch Gladbach, Germany). In brief, CD4⁺ T cells are isolated directly following PBMC isolation or after overnight storage at 4° C. in RPMI 1640 media supplemented with penicillin/streptomycin, glutamine and 10% fetal bovine serum (low Endotoxin FBS, GibcoBRL, Island, N.Y.). Hapten conjugated Ab specific for monocytes, granulocytes, CD8⁺ T cells, B cells, NK cells, platelets, and early erythroid precursors were added to PBMC in the standard MACS buffer of 2 mM EDTA and 0.5% BSA (Research Organics, Cleveland, Ohio) in PBS. After washing in MACS buffer, and incubation with anti-hapten Ab conjugated magnetic beads, the cells were washed again with MACS buffer and separated using the Possel_S program (single column, slow run) on AutoMACS separation columns (Miltenyi Biotec). Using this method, CD4⁺ T cells were routinely isolated to >90% purity and viability. CD45RA microbeads (Miltenyi Biotec) can be added during the incubation step with anti-hapten Ab conjugated magnetic beads, allowing removal of CD4⁺CD45RA⁺ (naive) T cells. This allows further enrichment for CD4⁺CD25⁺ cells (which we have determined are CD45RA⁻, CD45RO⁺). Purified CD4⁺ T cells can be isolated directly following PBMC isolation or from previously frozen PBMC (see above), in which case they are stored overnight in media at 1-2×10⁸ cells/ml at 4° C. Alternatively, PBMC may be similarly stored overnight with CD4⁺ T cells isolation performed the morning before cell sorting. Purified CD4⁺ T cells are then stained (as described above) with FITC-anti-CD25 and Cy-Chrome -anti-CD4 at a concentration of 5×10⁷-10⁸ cells/ml. For some experiments total CD4⁺ T cells are additionally stained with PE-anti-CD45RO to allow isolation of naive and memory CD4 T cell populations. Following staining, and washing with media, cells are resuspended in media at 3-6×10⁷ cells/ml and sorted at 4° C. into 1 ml of Endotoxin free FBS using a FACSVantage (Becton Dickinson) or MoFlo flow cytometer (Cytomation, Fort Collins, Colo.). Sorting gates are set to isolate CD4⁺CD25⁺ cells that represent ˜2% of total CD4⁺ T cells, or up to 4% if CD4⁺CD45RA⁺ (naive) T cells have been depleted. CD4⁺CD25⁻ cells are also sorted as a total population, or into CD4⁺CD25⁻CD45RO^{hi} and CD4⁺CD25⁻CD45RO^{low} populations for staining, in vitro functional assays, or RNA isolation. Using this method, we typically isolate viable CD4⁺CD25⁺ cells to ˜95% purity, and CD4⁺CD25⁻ cells to ˜99% purity.

The best mode for isolating CD4⁺CD25⁺ regulatory T cells utilizes PBMC isolated from leukopheresis product drawn on the same day, with isolation of CD4⁺ T cells such that sorting can be performed on the next day. Use of CD45RA microbeads allows further enrichment of CD4⁺CD25⁺ cells prior to sorting on a flow cytometer. Without the use of CD45RA microbeads, the most pure population of CD4⁺CD25⁺ regulatory T cells is isolated by setting the sort gates to obtain the top ˜2% of CD25⁺ cells (using FITC-anti-CD25), or a higher percentage if additional enrichment procedures are utilized.

Monocyte Isolation and Proliferation Assays. For antigen presenting cells, monocytes were isolated by negative magnetic bead depletion using the Monocyte Isolation Kit from Miltenyi Biotec according to the manufacturer's instructions and in a similar fashion described above for CD4⁺ T cell isolation. Typically this allows isolation of CD14⁺HLA⁻DR⁺ cells at >90% purity, with minimal (<0.5%) contamination with B, T or natural killer cells. Monocytes are generally used fresh in proliferation assays, but freezing protocols have also been developed (as described for PMBC above).

Proliferation assays were performed in a total of 200 μl RPMI-1640 media (see above) using U-bottom 96-well plates incubated at 37° C. with 5% CO₂. Typically 5×10⁴ CD4⁺CD25⁻ cells, 5×10⁴ CD4⁺CD25⁺ cells, or a mixture of 5×10⁴ CD4⁺CD25⁻ cells with titrated amounts of CD4⁺CD25⁺ cells were present with 1-2×10⁴ gamma irradiated (2000 rad) autologous monocytes, and stimulated with nothing; ConA 1 ug/ml (Sigma); or the anti-CD3 mAb OKT3 (affinity purified in our own facility on protein A sepharose). On day 3, [³H]thymidine (specific activity 20 Ci/mmol, NEN, Boston, Mass.) is added at 2.5 μCi/well for 8 hours, or sometimes overnight. Cells are then harvested and incorporated [³H]thymidine is measured by standard methods. Proliferation is measured by mean [³ H]thymidine incorporation in duplicate or triplicate wells +/−SEM.

RNA Isolation and RT-PCR Analysis. RNA was isolated from sorted populations of CD4 T cells using RNAeasy mini-spin columns (Quiagen Inc., Valencia, Calif.). To remove any contaminating genomic DNA, RNA was treated with treated with DNAse I using the DNA-free kit (Ambion Inc., Austin, Tex.). Random hexamer primed cDNA was generated using TaqMan Reverse Transcriptase Reagents (Perkin Elmer Applied Biosystems, Foster City, Calif.). RT-PCR reactions using TaqMan Universal PCR Master Mix (Perkin Elmer Applied Biosystems) were performed in triplicate wells of 96-well optical reaction plates using a ABI Prism 7700 machine according to the manufacturer's instructions using the following cycle parameters: 50° C.×2′ for 1 cycle, 95° C.×10′ for 1 cycle, 95° C.×15″ and 60° C.×1′ for 45 cycles (Perkin Elmer Applied Biosystems). Data described in this patent application utilized the following PCR primers and probes. Reactions were performed using cDNA corresponding to ˜1000-2000 cell equivalents per well. Oligonucleotides for PCR amplification were generated in house (at Immunex Corp.) by standard methods. Taqman analysis of genes of interest utilized 6FAM labeled reporters normalized to an internal VIC labeled β-actin standard (generated by Perkin Elmer Applied Biosystems). All probes were used at 100 nM, and primers at 300 nM except for β-Actin, in which case 30 nM primers were used. Sequences of primers and probes used for RT-PCR analysis are as follows:

β-Actin: primers: (SEQ ID NO:1) 5′-CCTGGCACCCAGCACAA-3′ and (SEQ ID NO:2) 5′-GCCGATCCACACGGAGTACT-3′; probe: (SEQ ID NO:3) 5′-ATCAAGATCATTGCTCCTCCTGAGCG-3′.

CD4: primers: (SEQ ID NO:4) 5′-GGAAATCAGGGCTCCTTCTTAAC-3′ and 5′-(SEQ ID NO:5) GTCCCAAAGGCTTCTTCTTGAG-3′; probe: (SEQ ID NO:6) 5′-CCATCCAAGCTGAATGATCGCGCT-3′.

CD25: primers: (SEQ ID NO:7) 5′-CGATGACCCGCCAGAGAT-3′ and (SEQ ID NO:8) 5′-CATTCACAGTTCAACATGGTTCCT-3′; probe: (SEQ ID NO:9) 5′-CCACACGCCACATTCAAAGCCATG-3′.

CTLA-4: primers: (SEQ ID NO:10) 5′-CGCCAGCTTTGTGTGTGAGT-3′ and (SEQ ID NO:11) CCTGCCGAAGCACTGTCA-3′; probe: (SEQ ID NO:12) 5′-TGCATCTCCAGGCAAAGCCACTGA-3′.

IL-10: primers: (SEQ ID NO:13) 5′-CGGCGCTGTCATCGATTT-3′ and (SEQ ID NO:14) 5′-TGGAGCTTATTAAAGGCATTCTTCAC-3′; probe: (SEQ ID NO:15) 5′-TCCACGGCCTTGCTCTTGTTTTCACA-3′.

Probes and primers for human CD30, OX40 and 4-1BB were similarly designed (using Primer Express software, Perkin Elmer Applied Biosystems) and used for RT-PCR analysis. Analysis of results was performed using ABI Prism Sequence Detector Software. Relative gene expression is normalized to the internal β-Actin standard for each well, and then subsequently normalized to the amount of CD25 present in one sample of CD4⁺CD25⁻ cells (an arbitrary value to facilitate comparison of expression of different genes on the same graph). Data is presented as the mean of triplicate wells +/− the standard deviation.

Results

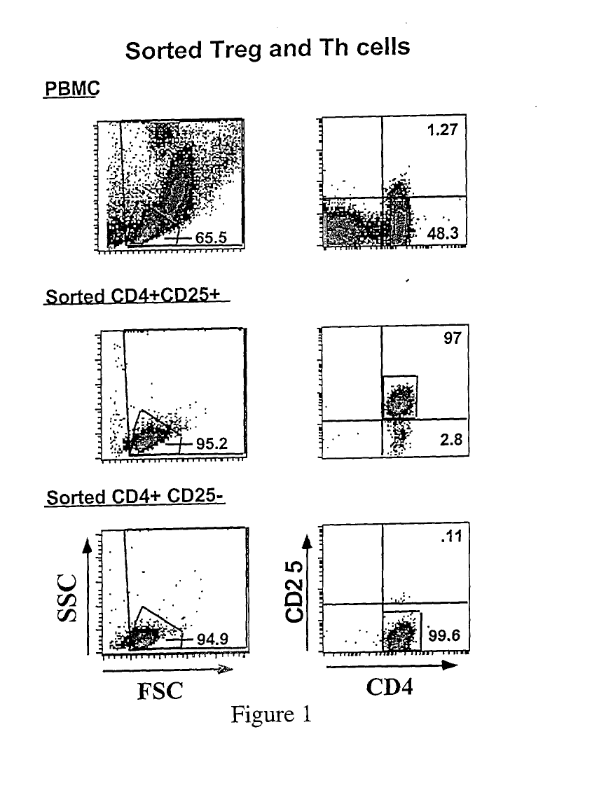

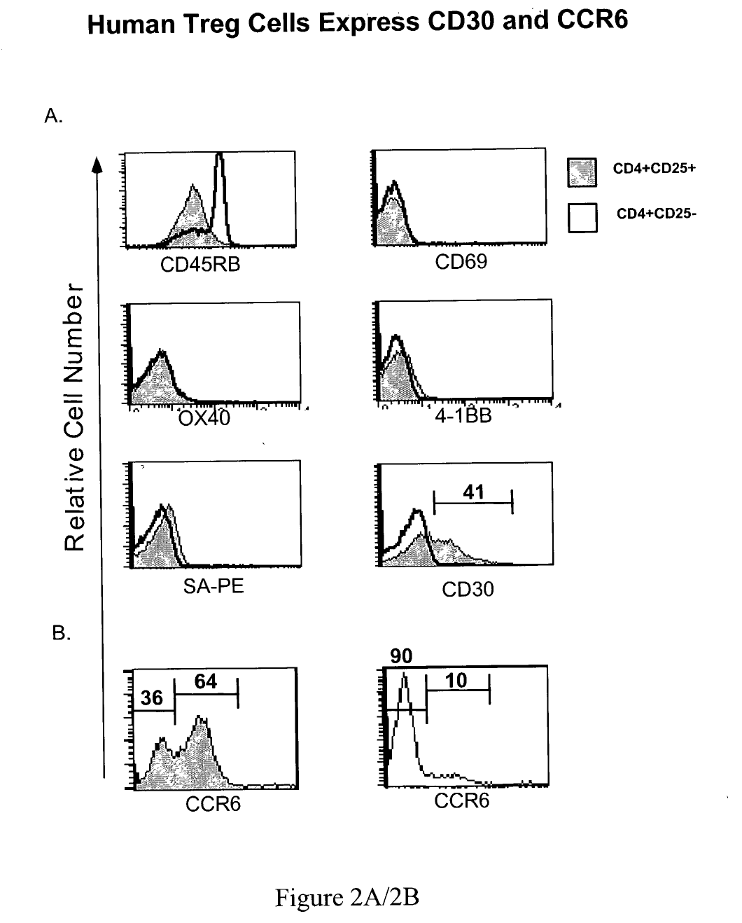

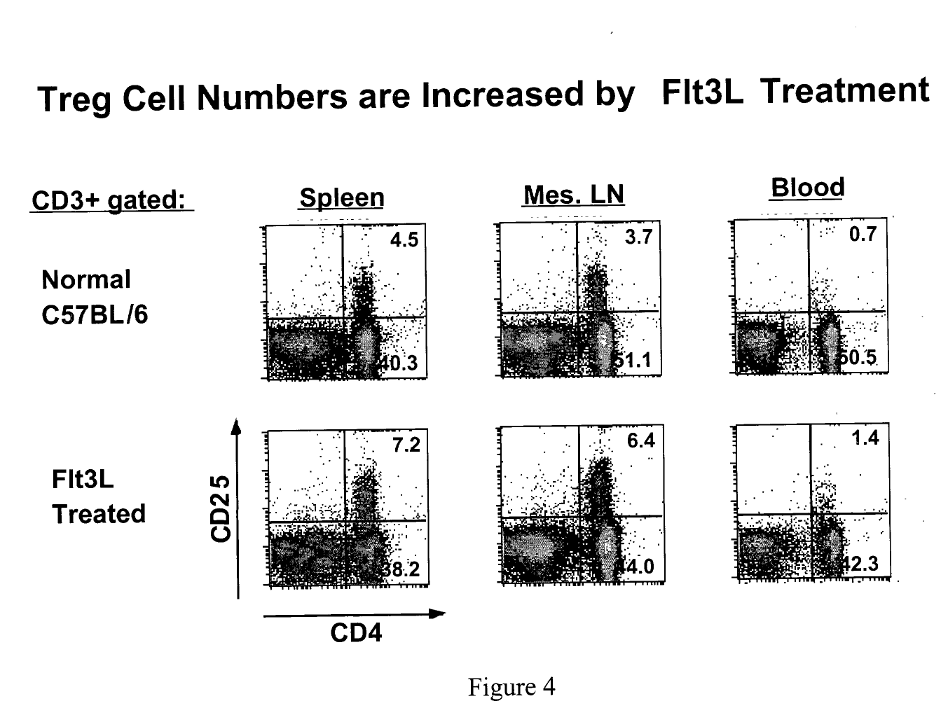

Initial studies have been aimed to determine whether a population can be defined of CD4⁺CD25⁺ Treg cells in human peripheral blood that is analogous to murine CD4⁺CD25⁺ cells. Although murine Treg cells are generally isolated from spleen and lymph node, we chose to study Treg cells in human PBMC preps because they are readily available. Four color flow cytometry showed that about 0.5-2% of human PBMC (or 1-4% of CD4 T cells, >10 donors analyzed to date) are CD25⁺. These cells are CD69⁻CD45RB^{lo} (see FIGS. 1 and 2A) and CD62L^{hi}, consistent with the phenotype of Treg cells observed in mice. A similar population of CD8⁺CD25⁺ was not present in human PBMC suggesting that staining of the CD4⁺CD25⁺ cells was specific. However the staining pattern of human CD4⁺CD25⁺ cells in peripheral blood was somewhat different than that for murine CD4⁺CD25⁺ Treg cells in spleen and lymph node, in that the murine CD25⁺ cells stained more brightly and were more abundant (5-10% of CD4⁺ T cells). We tried multiple staining methods for CD25 (including PE conjugated Ab, and two step staining using biotinylated anti-CD25 mAb) to determine whether we could shift the staining of CD25 on CD4+ T cells to give a more clear separation from the CD4+CD25− T cells. However, the best mode for staining utilized FITC- or APC-anti-CD25 and Cy-Chrome-anti-CD4, because other methods increased background staining of CD25⁻ cells. In addition we have also stained murine blood and observed that murine blood, like human blood contains a lower percentage of CD25⁺ cells (see Murine Treg Studies below, FIG. 4).

Next we developed a protocol to isolate these putative human Treg cells. Using negative bead depletion of ficoll purified human PBMC leukopheresis preps, we routinely obtain human CD4⁺ T cells at ˜95% purity. These cells are then stained with anti-CD4 and anti-CD25 mAb and sorted on the flow cytometer. In a typical prep 10⁹ PBMC yield 2-5×10⁸ CD4⁺ T cells which are sorted to give 1-3×10⁶ CD4⁺CD25⁺ cells (>95% pure) and an excess (>5×10⁷) of CD4⁺CD25⁻ responder cells. In some experiments the cells are also stained for CD45RO and the CD25⁻-cells are sorted into CD45RO^{hi} and CD45R^{lo} populations. The CD4⁺ T cells do not appear to become activated by any of our experimental manipulations as assessed by altered CD25 or CD69 expression. Flow cytometry of sorted CD4⁺CD25⁺ cells showed that they are CD69⁻, CD62L^{hi}, CD45RB^{low}, CD45RO^{hi}. The latter two markers are consistent with the memory phenotype profile observed for murine Treg cells (CD45RB^{low} and CD44^{hi}). We have also determined that gated human CD4⁺CD25⁺ cells are also CD45RA⁻, and further enriched for the CD4⁺CD25⁺ cells prior to sorting using CD45RA conjugated microbeads to deplete CD25⁻ naive T cells. Thus, by phenotypic criteria, we have defined a population of CD4⁺CD25⁺ Treg cells in human peripheral blood that is analogous to murine CD4⁺CD25⁺ cells.

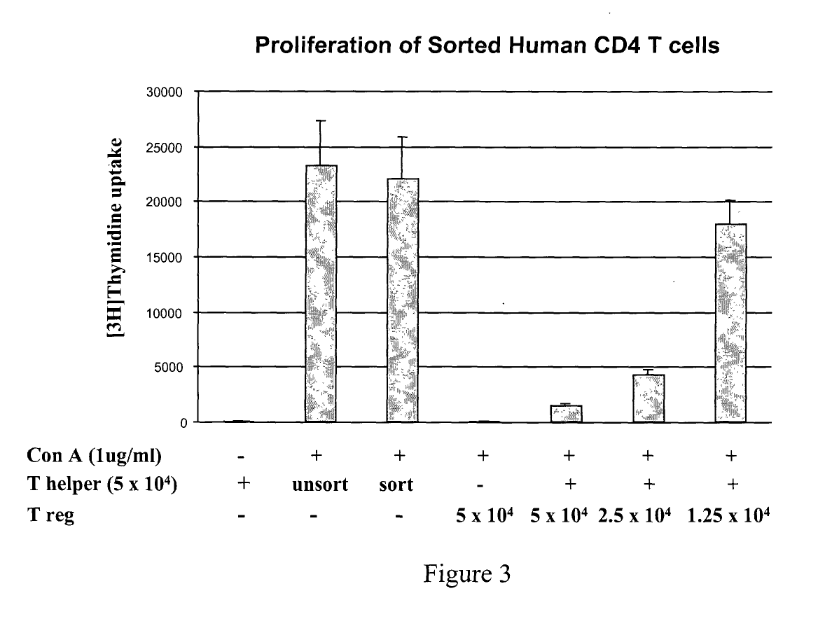

Next we performed in vitro proliferation assays to determine whether human CD4⁺CD25⁺ cells were functionally similar to murine Treg cells (FIG. 3). Sorted CD4⁺CD25⁺ cells were compared to CD4⁺CD25⁻ cells for their ability to proliferate in culture to autologous monocytes in the presence of ConA (1 ug/ml). Like murine Treg cells, human CD4⁺CD25⁺ cells were anergic (failed to proliferate) while normal CD4⁺ Th cells proliferated well. Importantly, in a dose dependent manner human CD4⁺CD25⁺ cells inhibited the proliferation of CD4⁺CD25⁻ cells, with >90% inhibition when mixed in a 1:1 ratio. These findings have been reproduced (n=3 assays using different donors), and demonstrate that human CD4+CD25+ T cells isolated from PBMC behave similarly to murine Treg cells isolated from spleen and lymph node.

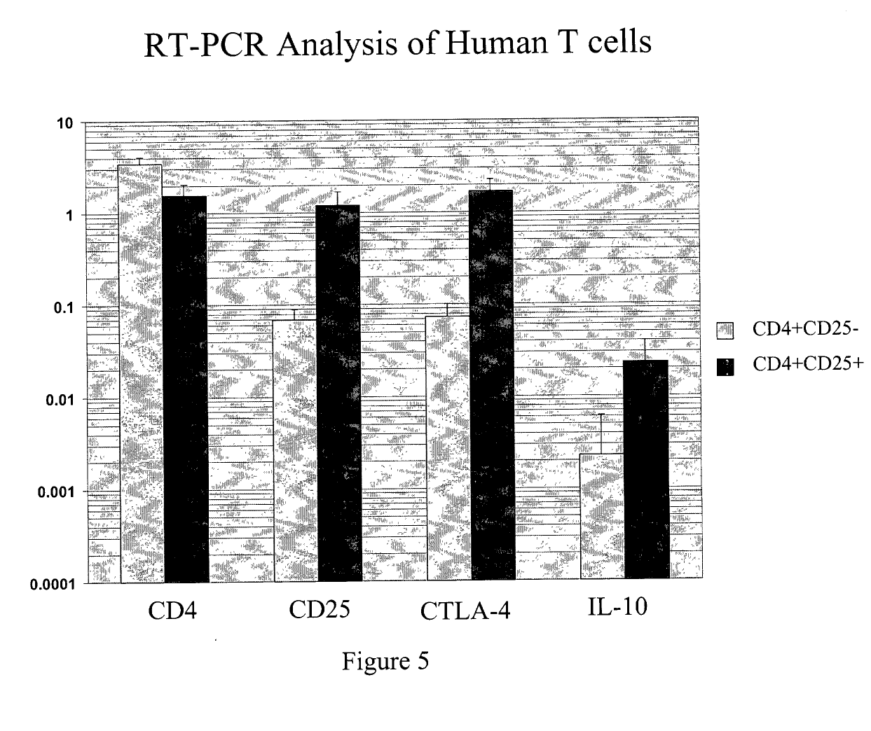

To further characterize the human CD4⁺CD25⁺ regulatory T cells, we have performed RT-PCR studies to determine whether they show increased mRNA expression of other molecules present in murine Treg cells. As expected CD4⁺CD25⁺ T cells expressed comparable amounts of CD4 to that of CD4⁺CD25⁻ cells, and about 20-fold greater amounts of CD25 mRNA, consistent with the purity of the sorted cells (FIG. 5). Notably, human CD4⁺CD25⁺ T cells also expressed 20-fold or 10-fold more mRNA for CTLA-4 and IL-10, respectively. CTLA-4 and IL-10 are specifically expressed by murine Treg cells, and have been shown to be important for their function. Thus by phenotypic and functional criteria, we have demonstrated that we can isolate a population of human regulatory T cells from peripheral blood that is analogous to murine CD4⁺CD25⁺ regulatory T cells.

We have initiated gene discovery efforts to understand Treg biology and identify therapeutic targets expressed by this cell population. To perform array analysis, isolated human Treg cells (CD4⁺CD25⁺), will be compared to memory CD4⁺ T cells (CD4⁺CD25⁻CD45RO^{hi}), and to naive CD4⁺ T cells (CD4⁺CD25⁻CD45RO^{lo}). This will increase the power of the array on Treg cells, which express a number of memory cell markers. Human CD4⁺ T cells have been successfully sorted into these populations in numbers sufficient for array analysis.

As part of our studies to characterize these human Treg cells we have performed RT-PCR analysis of known genes of interest in murine Treg (see below). These studies revealed that relative to Th (CD4⁺CD25⁻), murine Treg (CD4⁺CD25⁺) showed increased mRNA for the TNFR family members OX-40, CD30, and 4-1BB. As a follow up to these studies we performed flow cytometry to determine expression of these molecules on our human CD4⁺CD25⁺ regulatory T cells. Notably, we have determined that human Treg (unlike CD4⁺CD25⁻ cells) expressed cell-surface CD30 (see FIG. 2A), but not detectable OX-40 or 4-1BB. As a control, we were able to detect OX-40 and 4-1BB on PHA activated human T cells. In addition we have performed RT-PCR analysis of CD30, OX40 and 4-1BB expression in sorted human CD4⁺CD25⁺ regulatory T cells vs. CD4⁺CD25⁻ cells, CD4⁺CD25⁻ CD45RO^{hi} (memory) and CD4⁺CD25⁻CD45RO^{lo} (naive) and found that CD30 and 4-1BB mRNA is increased >20-fold in human Treg relative the other CD4⁺T cell populations. In contrast to murine Treg, human Treg do not show increased OX40 mRNA or protein expression. These results have been confirmed using independent donors. Thus human peripheral blood derived Treg constituitively express CD30.

In addition we have found that human CD4⁺CD25⁺ regulatory T cells express cell surface CCR6. As discussed below, we discovered that murine regulatory T cells have increased CCR6 mRNA and decreased CCR7 mRNA relative to murine Th cells. As a follow up to these studies, we stained human CD4⁺CD25⁺ regulatory T cells and found that they predominantly express CCR6, unlike CD4⁺CD25⁻ cells, which are predominantly CCR6 negative. (FIG. 2B). In one experiment we also observed that human Treg are predominantly CCR7 negative. Thus the protein expression data for human Treg and Th cells correlates with the RT-PCR data for the analogous murine populations.

### EXAMPLE 2

### Murine Treg Studies

Materials and Methods

Antibodies and flow cytometry: The following anti-murine mAb were purchased from BD Pharmingen: Cy-Chrome-anti-CD4, PE- or APC-anti-CD25 (PC61), FITC-anti-CD44, PE- or Biotin-anti-CDw137 (4-1BB, Ly63), PE-anti-CD30, and Biotin-anti-OX40L (Rm134L). Affinity purified FITC-anti-4-1BB (M6), FITC-anti-CD30L (M15), FITC-anti-CD40L (M158), and FITC-anti-OX40 (M5) were generated in house. Staining was performed by standard methods on ice in PBS containing 5% FBS in the presence of the anti-Fc receptor mAb 2.4G2 (from BD Pharmingen or affinity purified in house). Samples were washed, fixed in 1% paraformaldehyde and analyzed as described above.

Isolation of CD4+ memory, naive and regulatory T cells. Murine CD4⁺ T cell subsets are isolated in a similar manner to that described above for human CD4⁺ T cells using negative magnetic bead selection to enrich for CD4⁺ T cells, and sorting of specific subsets using mAb to CD4 and CD25. For studies described in this application, CD4⁺ T cells were isolated from adult C57BL/6 (Jackson Laboratories, Bar Harbor, Me.) or DO11.10 T cell receptor transgenic mice (described in Murphy et al (1990) Science 250:1720 and bred in house) housed in our specific pathogen free (SPF) facility. Typically, spleen and lymph nodes are harvested from 10-15 mice and disaggregated by standard methods. Following red blood cell lysis, CD4⁺ T cells are isolated by negative magnetic bead selection using the Murine CD4⁺ T cell enrichment cocktail (Stem Cell Technologies, Vancouver, Canada), and separated on AutoMACS columns (Miltenyi Biotec) as described for isolation of human CD4⁺ T cells. Purified murine CD4⁺ T cells are then stained with Cy-Chrome-anti-CD4 and PE-anti-CD25. For some experiments, total CD4+ T cells are additionally stained with FITC-anti-CD44 to allow sorting of memory (CD44^{hi}) and naive (CD44^{lo}) T cells. Cells are sorted into 1 ml FBS. Using this method, we typically isolate viable CD4⁺CD25⁺ cells to >95% purity, and CD4⁺CD25⁻ cells to ˜99% purity.

Flt3-L treatment of mice. Mice are treated by intraperitoneal injection with 10 ug per day of recombinant soluble human Flt3-L in PBS, or PBS alone as control for the indicated period of time. Flt-3L treatment is known to expand multiple dendritic cell populations in vivo (Marakovsky et al. (1996) J. Exp. Med. 184:1953-62).

Proliferation Assays. Proliferation assays were performed in a total of 200 μl IMDM media supplemented with 5% FBS (Hyclone, Logan, Utah) and 2-mercaptoethanol (GibcoBRL) using U-bottom 96-well plates incubated at 37° C. with 5% CO₂. Typically 10⁵ CD4⁺CD25⁻ cells, 10⁵ CD4⁺CD25⁺ cells, or a mixture of 10⁵ CD4⁺CD25⁻ cells with titrated amounts of CD4⁺CD25⁺ cells were present with 3×10⁵ gamma irradiated (3000 rad) red blood cell lysed splenocytes as antigen presenting cells, and stimulated with nothing, or 10 ug/ml anti-CD3 mAb 500-A2 (affinity purified in house). In some cases APC are stimulated with E. coli LPS (Sigma) at 10 μg/ml for two days and then washed prior to use in proliferation assays. Alternatively, CD4⁺ T cells from DO11.10 T cell receptor transgenic mice are stimulated with titrated amounts of ovalbumin peptide (Ova 323-339 synthesized and HPLC purified in house). On day 3 cells harvested with 2 μCi/well [³H]thymidine present during the last 16 hr. of culture.

RNA Isolation and RT-PCR Analysis This was performed in a manner similar to that described above for human CD4⁺ T cells. PCR primers (generated in house) and probes (generated by Perkin Elmer Applied Biosystems) for murine gene sequences described in the results section were designed using Primer Express Software (Perkin Elmer Applied Biosystems) according to the manufacturer's instructions. Data Analysis was performed as described for human RT-PCR studies with the relative gene expression normalized to the internal murine β-actin standard for each well and then to the amount of CD4 cDNA present in the CD4⁺CD25⁻ population as an arbitrary value to allow representation of multiple genes on a single graph.

Results

Studies of murine Treg cells have been aimed toward: 1) establishing in vitro assay systems to measure Treg cell activity, 2) Taqman and flow cytometry analysis to identify regulatory molecules specific to Treg cells, and 3) experiments to analyze the effect of specific DC subsets on Treg cell proliferation and effector function and 4) analysis of the effect of immunodulating agents on Treg expansion, depletion, or activation in vivo. We have developed a protocol to isolate Treg cells for routine in vitro studies. Typically, spleens and lymph nodes are harvested from 10 mice, and about 2×10⁸ CD4⁺ T cells are isolated at ˜95% purity by negative magnetic bead depletion. This step is followed by staining for CD4 and CD25 and cell sorting to give 10⁷ CD4⁺CD25⁺ Treg cells and an excess of CD4⁺CD25⁻ responder Th cells. The isolated Treg cells behave as expected from published reports (see A. M. Thorton and E. M. Shevach (1998) J.E.M. 188:287-296) in that they are anergic to proliferative stimuli such as anti-CD3 mAb or ConA+antigen presenting cells (APC, RBC lysed irradiated congenic splenocytes) and inhibit the proliferation of CD4⁺CD25⁻ Th cells in co-culture experiments.

Real time PCR (Taqman) was performed to analyze expression of known gene products focusing on TNFR-family members and their ligands, CD28-family members and chemokine receptors. As Treg cells are thought to inhibit the response of Th cells by a cell-contact dependent mechanism (Sakaguchi (2000), supra.; Shevach (2000), supra.), TNFR/TNF family members are likely players in the control of Treg responses or effector function. By Taqman analysis, relative to CD4⁺CD25⁻ cells, murine Treg cells showed similar levels of CD4, and increased CD25, IL-10 and CTLA-4 mRNA, as expected. Of the TNFR family members examined, OX-40, 4-1BB, and CD30 mRNAs were markedly (10-30×) increased in Treg cells relative to Th cells, and this result was consistent for CD4⁺ T cells isolated from both spleen and lymph node. RANK was up only 5× in Treg cells relative to Th cells, and CD27 mRNA levels were similar between the two populations. Of the TNF family members tested (including TNFα, OX40L, CD30L, CD40L, RANKL), only CD40L was significantly different with a 5× decrease in Treg cells in one experiment. Aside from CTLA-4, other CD28-family members (CD28, ICOS) showed little difference in expression between Treg and Th cells. Analysis of chemokine receptors was notable in that Treg showed increased expression of CCR6 (6 fold) and decreased expression or CCR7 (4 fold) relative to CD4⁺CD25⁻ cells. The majority of CD4⁺ T helper cells normally express CCR7. This may be of particular functional significance as there is a switch from CCR6 to CCR7 expression as dendritic cells mature (see Dieu et al. (1998) J. Exp. Med. 188:373-386, for example) and that CCR6 mediates dendritic cell localization to mucosal tissue (see Cook et al. (2000) Immunity 12:495-503). These results suggest that Treg may preferentially home to sites that contain immature DC or possibly mucosal DC.

Flow cytometry was performed to analyze whether differences in mRNA were consistent with cell-surface protein expression. Treg cells did not show significant cell surface expression of 4-1BB, CD30, CD30L, or OX-40L. However, OX-40 was significantly increased. Increased OX-40 expression was specific to Treg cells in that memory CD4⁺CD25⁻ T cells (CD44^{hi} gated) were OX-40⁻. Based on these results (as discussed above), we also stained human CD4⁺CD25⁺ cells for 4-1BB, CD30, and OX-40 expression, but unlike murine Treg cells, our results indicate that unstimulated human Treg cells only express CD30 and not OX-40. These differences may be due to species differences or possible due to differences in the tissue source, as murine Treg are isolated from spleen and lymph node, while the human Treg we have analyzed come from blood. We have demonstrated that relative to spleen and lymph node, Treg in murine blood are less abundant (see FIG. 4), and in this way resemble CD4/CD25 staining profiles of the human CD4+CD25+ regulatory T cells described above.

We have hypothesized that molecular targets may expand or reduce other cell populations that impact Treg numbers or activity. For example, agents may control the expansion of specific DC subsets and thereby alter immune tolerance mediated by Treg cells. To investigate this possibility, we have analyzing changes in Treg cell numbers/phenotype in Flt3-L treated mice. In Flt3-L treated mice, the absolute number of Treg as well as the ratio of Treg/Th is increased (see FIG. 4). This finding has been highly reproducible in 4 different experiments. For example, absolute Treg cell numbers increased 3 fold or 6 fold in spleen and lymph node (respectively) from C57BL/6 mice treated with Flt3-L for 17 days. While absolute numbers of CD4+CD25− cells also increased following Flt3-L treatment, Treg cells appear to be preferentially expanded by Flt3-L because the ratio of CD4⁺CD25⁺ to CD4⁺CD25⁻ cells is increased following Flt3-L treatment (˜50% increase in the ratio in spleen, ˜30% in mesenteric lymph node, and ˜70% in blood). Like normal Treg cells, Treg cells from Flt3-L treated mice are anergic in vitro (under conditions that would normally stimulate proliferation of T helper cells) and show a similar capacity to inhibit the proliferation of CD4⁺CD25⁻ cells in co-culture experiments. In fact, for more recent experiments, Flt3-L treatment has been used to increase the number of Treg cells obtained for in vitro assays.

### Example 3

### Effect of Antigen Dose

To further understand the control of regulatory T cell activity, we performed in vitro experiments in which CD4⁺25⁺ and CD4⁺CD25⁻ T cells from T cell receptor (Tcr) transgenic mice are stimulated with titrated doses of specific antigenic peptide, by spleen-derived antigen-presenting cells (APC).

Materials and Methods

Mice and Flt3-ligand (Flt3-L) treatment. All mice were housed at Immunex Corp. (Seattle, Wash.) under specific pathogen-free conditions. DO11.10 mice, whose T cells bear transgenic TCR specific for chicken ovalbumin peptide fragment 323-339 (OVA) and I-A^{d} (28), were bred and maintained at Immunex. [BALB/c x A]F1 (CAF1) mice (H-2^{d/k}) and AND mice, whose T cells bear transgenic TCR specific for pigeon cytochrome c peptide fragment 88-104 (PCC) and I-E^{k} (29), were from Jackson Laboratory (Bar Harbor, Me.). BALB/c mice were from Charles River Laboratories (Wilmington, Mass.). Flt3-L-treated mice were injected substantially as described above.

mAbs and flow cytometry. The following conjugated mAbs were obtained from BD Pharmingen (San Diego, Calif.): APC-anti-CD3 and CD45R/B220; biotin-anti-CD19; Cy-Chrome- or PerCP-Cy5.5-anti-CD4 and CD8α; FITC-anti-CD19, CD62L, CD69, CD80, CD86 and Gr-1; PE-anti-CD11c, and CD25 (PC61). FITC-conjugated isotype controls were: rat IgG₂ₐ (R35-95) for CD62L and CD86; hamster IgG group 1 (A19-3) for CD69; and hamster IgG group 2 (B81-3) for CD80. Anti-Fc receptor CD16/32 (2.4G2) was affinity purified at Immunex. Single cell suspensions were incubated in PBS containing 5% FCS (HyClone, Logan, Utah) and 2.4G2, followed by conjugated mAbs. Staining with biotinylated mAbs utilized Cy-Chrome-labeled streptavidin (BD Pharmingen) as a second step reagent. Following staining, cells were washed and fixed in PBS containing 1% paraformaldehyde. Flow cytometry was performed on a FACSCalibur machine and analyzed using CellQuest software (Becton Dickinson, San Jose, Calif.).

Isolation and preparation of APC populations. RBC-lysed BALB/c splenocytes were washed twice and resuspended in complete IMDM for use directly in proliferation assays (in the case of whole spleen APC) or enriched for B cells by negative immunomagnetic selection. Briefly, non-B cells were labeled using the Murine B Cell Enrichment Cocktail (Stem Cell), then passed over a column inside a VarioMACS. Greater than 97% of cells eluted in the flow-through fraction were CD19⁺. Activated whole spleen or B cells were obtained following 2 days of culture in 10 μg/ml LPS (Sigma). Whole spleen or B cell APC were irradiated (3000 rads from a ¹³⁷Cs source) prior to use in proliferation assays. For DC-enriched APC, single spleen cell suspensions from Flt3-L-treated mice were subjected to Nycodenz gradient centrifugation (Invitrogen), followed by immunomagnetic positive selection of Nycodenz-buoyant cells using CD11c microbeads (Miltenyi Biotec) and an AutoMACS cell separator (Miltenyi Biotec). Greater than 97% of eluted cells were CD11c⁺. Alternatively, Nycodenz-buoyant cells were stained with PE-anti-CD11c and APC-anti-B220, then separated into CD11c^{bright}B₂₂₀⁻ (cDC) and CD11c^{dim}B220⁺ (pDC) using the MoFlo cell sorter. DC were either used fresh or cultured overnight in 10 μg/ml LPS for whole CD11c⁺ DC or 1 μg/ml CpG oligodeoxynucleotide (TCCATGACGTTCCTGACGTT, SEQ ID NO:16; Sigma-Genosys, The Woodlands, Tex.) with or without 5 μg/ml murine CD40L (Immunex Corp.) for sorted DC. To promote cell survival, 20 ng/ml murine GM-CSF (Immunex Corp.) was included in DC cultures.

Proliferation assays. Th (2.5×10⁴), Treg (2.5×10⁴), APC (7.5×10⁴ whole spleen or B cell, 1.25×10⁴ CD11c⁺ DC, or 5×10³ specific DC subset), and peptide (5 nM-5 μM OVA, 10 μM PCC) in complete IMDM were added to 96-well U-bottom plates (Corning, Corning, N.Y.) for a total of 200 μL/well. Except where otherwise noted, 2.5×10⁴ of indicated T cell populations were plated per well. OVA and PCC were synthesized and purified by HPLC at Immunex. Plates were incubated for 96 hours at 37° C., 5% CO₂, with 1 μCi [³H]TdR (Amersham Biosciences, Piscataway, N.J.) added per well during the last 24 hours of the assay. Contents of each well were transferred to Filtermat A glass fiber filters (Wallac, Turku, Finland) using the Brandel harvester (Brandel, Gaitherburg, Md.) and read on a TriLux 1450 MicroBeta counter (Wallac). In some experiments, 50 μL of supernatant was harvested from each well at 72 h of culture for cytokine analysis.

Analysis of cytokine production. Levels of IL-2, IL-4, IL-10, and IFN-γ in 72 h culture supernatants from T cell proliferation assays were determined using the Beadlyte Mouse Multi-Cytokine Detection System (Upstate Biotechnology, Lake Placid, N.Y.) and the Luminex¹⁰⁰ plate reader (Luminex Corporation, Austin, Tex.) according to manufacturers' instructions. Quantification of cytokines was performed by regression analysis from a standard curve generated from cytokine standards included in the kit. Lower limits of detection were: 2 pg/ml for IL-2, 0.2 pg/ml for IL-4, 35 pg/ml for IL-10, and 3 pg/ml for IFN-γ.

Statistical analysis. Statistical analysis (unpaired t test) was performed using InStat software (GraphPad Software Inc., San Diego, Calif.).

Results

Treg cells from TCR transgenic mice activated by peptide and splenic APC efficiently suppress antigen-specific Th proliferative responses. In the spleen, several potential cell types can serve as APC, each with different capacities to stimulate T cell function. To determine whether different spleen-derived APCs vary in their capacity to support Treg function, we compared the ability of freshly isolated whole splenocytes, B cells or DC to elicit peptide specific regulation of Th proliferation in vitro. Treg cells were anergic to a wide range of OVA doses presented by unstimulated whole spleen (containing B cells, DC, and macrophages), B cell or CD11c⁺ DC APC. Furthermore, Treg cells interacting with whole spleen or B cell APC potently suppressed Th cell proliferation across a wide range of OVA doses. Thus, B lymphocytes elicit potent regulation by CD4⁺CD25⁺ T cells in vitro, even though they are less efficient at stimulating Th proliferative responses. DC also strongly supported regulation at lower peptide doses. This finding is similar to that in a previous report showing Treg suppressor function at OVA doses up to 100 nM using unactivated spleen cell APC. However, increasing the antigen dose presented by DC resulted in complete loss of Treg-mediated suppression. Taken together, our results indicate that Treg-mediated suppression is lost with more potent antigen presentation.

We next analyzed the efficiency of regulation elicited by activated whole spleen APC, B cells (cultured with LPS), and DC (cultured with LPS and GM-CSF). Activation resulted in upregulation of CD80 and CD86 expression on all three APC types, most dramatically on DC. As expected with activation, all three types of APC induced more vigorous peptide-specific Th cell proliferative responses. Treg cells, on the other hand, were anergic to a wide range of OVA doses presented by all three types of activated APC. Notably, Treg cells interacting with activated B cells (as with unactivated B cells) potently suppressed Th cell proliferation across all OVA doses tested. In contrast, Treg cells interacting with activated whole spleen APC exhibited a peptide dose-dependent loss of regulatory activity, and suppression was lost at a log lower peptide dose when activated instead of unactivated DC were used as APC. Thus the ability of Treg cells to suppress Th proliferation in vitro is favored under poor stimulatory conditions (such as low antigen dose presented by unactivated B cells), but is lost during conditions of potent stimulation (such as high antigen dose presented by activated DC).

pDC preferentially support Treg activity at a high peptide dose. The murine spleen contains several functionally and phenotypically distinct DC subsets. We tested whether sorted CD11c^{bright}B220⁻ cDC and CD11c^{dimt}B220⁺ pDC subsets isolated from Flt3-L-treated mice differed in their capacity to stimulate Treg function in vitro. Treg cells were poorly proliferative in response to antigen presented by either DC. Notably, pDC supported Treg-mediated suppression at up to a log lower peptide dose compared to cDC. Although CD11c^{bright}CD8α⁺ and CD11c^{bright}CD8α⁻ cDC direct Th1 and Th2 responses, respectively, we noted no difference in the ability of these cells to support antigen-specific suppression.

We also analyzed the ability of activated cDC and pDC to support regulation. Since human pDC express TLR9 (CpG specific) but not TLR4 (LPS specific), and human and mouse pDC secrete IFN-α in reponse to CpG, we chose to activate the DC subsets with CpG instead of LPS. Culture with GM-CSF and CpG with or without CD40L resulted in increased CD80/86 levels on both subsets, more dramatically on cDC than pDC. Following activation, cDC but not pDC induced more vigorous peptide-specific Th proliferation. Treg-mediated suppression was lost at a lower peptide dose when activated instead of unactivated cDC were used as APC. In contrast, activation did not dramatically alter the ability of pDC to support suppression. These studies indicate that pDC, even in an activated state, preferentially support Treg cell activity.

Potently stimulated Th cells are refractory to suppression by functionally activated Treg cells. As shown above, Treg-mediated suppression is lost in the presence of high doses of antigen and activated APC in vitro. It is therefore possible that potent stimulation renders Treg cells non-functional. To test this possibility, we analyzed cytokine secretion by Treg and/or Th cells responding to OVA and activated splenic APC. Although Treg cells failed to secrete IL-2, IFN-γ, or IL-4, they exhibited a dose-dependent increase in IL-10 secretion, demonstrating a functional response to high peptide dose presented by activated APC. Furthermore, levels of IL-2, IL-4, and IFN-γ in OVA-stimulated cultures were significantly reduced by the presence of Treg cells. Notably, Treg cells did not suppress Th proliferation under the same conditions. Thus, potently stimulated Treg cells are functional, but fail to regulate Th proliferation. Although IL-2 levels in Th cultures were markedly decreased by the presence of Treg cells, significant amounts of IL-2 were present in co-cultures stimulated with 0.5 and 5 mM OVA (16.9 and 63.3 ng/ml, respectively). Other groups have demonstrated that addition of as little as 3 ng/ml IL-2 will override Treg-mediated suppression of Th proliferation in vitro. Thus, these results suggest that potently stimulated Th cells may produce sufficient levels of IL-2 to override Treg-mediated suppression.

We have demonstrated that Treg cells fail to suppress Th proliferation under conditions of potent stimulation. To distinguish whether this failure to suppress at high peptide doses reflects a deficit in Treg function or if highly stimulated Th cells are refractory to Treg functions, we developed a two-antigen regulation assay. This assay allows independent titration of specific peptide to Th cells and Treg cells, as previously described (see, for example, Thornton, and Shevach, 1998 and 2000, supra; Read et al., Eur. J. Immunol 28:3435, 1998, and Takahashi et al., Int. Immunol. 10:1969, 1998).

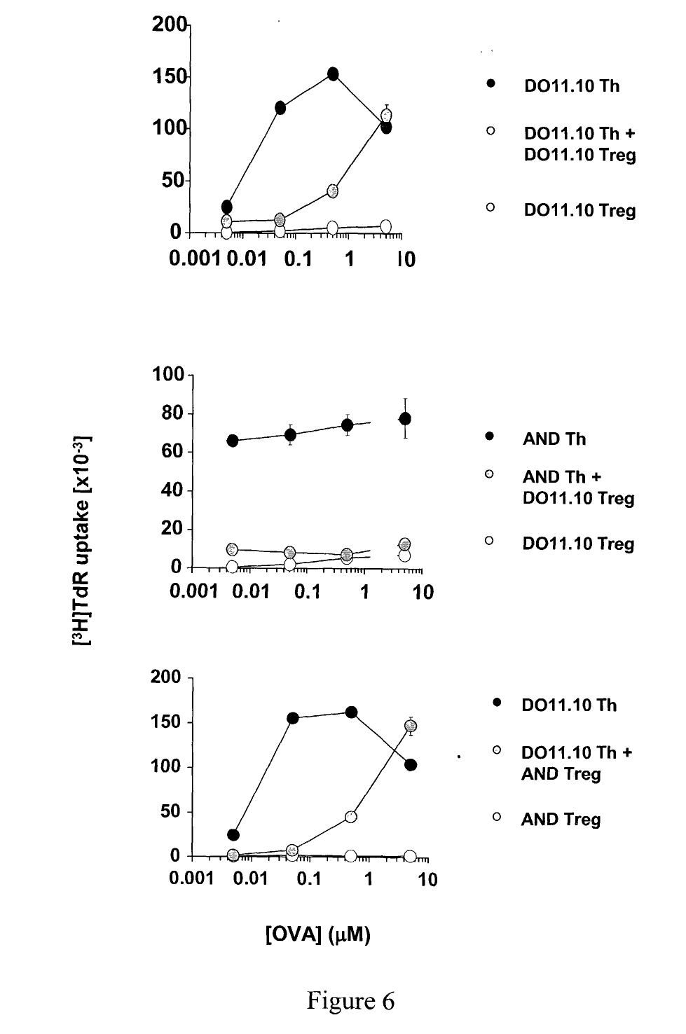

In our assay, DO11.10 (I-A^{d} and OVA-specific) and AND mice (I-E^{k} and PCC-specific) were used as sources for Treg and Th cells. For APC we used activated CAF1 (I-A^{d}/I-E^{k}-expressing) splenocytes capable of presenting antigen to both types of transgenic T cells. As expected, DO11.10 Treg cells effectively suppressed Th proliferation to OVA presented by activated CAF1 splenocytes at low but not high dose (FIG. 6, top panel). In contrast, DO11.10 Treg cells stimulated with high doses of OVA efficiently suppressed PCC-specific Th proliferation (FIG. 6, middle panel). Thus, Treg cells stimulated with high OVA peptide dose and activated APC retain the ability to suppress Th proliferation. We also analyzed the functional activity of Treg cells from AND mice, and determined that they efficiently suppress AND Th proliferation to 10 μM PCC and activated APC. This PCC dose was chosen because it was a mid-optimal dose for stimulating Th cell proliferation. PCC-activated AND Treg cells suppressed DO11.10 Th cell proliferation at lower (5-50 nM) OVA doses (FIG. 6, bottom panel). However, they failed to suppress the DO11.10 Th response to high (5 μM) OVA dose. Thus, with sufficient stimulation, Th cells can escape Treg-mediated suppression of proliferation.

Identification of reagents that modulate regulatory activity in vivo will likely be important for the treatment of autoimmune disease (by stimulation of the regulatory state) or cancer (to decrease the regulatory state and thereby allow enhanced anti-tumor immunity).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for the isolation of human regulatory T cells, the method comprising:
obtaining a cell sample comprising human T regulatory cells from a human donor;
contacting said cell sample with reagents that specifically recognize CD4, and CD25;
selecting for those cells that are CD4⁺CD25⁺, to provide an enriched population of regulatory T cells.

2. The method according to claim 1, wherein said regulatory T cells are characterized as CD69⁻.

3. The method according to claim 1, wherein said regulatory T cells are characterized as CD30⁺.

4. The method according to claim 1, wherein said regulatory T cells are characterized as CCR6⁺.

5. The method according to claim 1, wherein cell sample is a blood sample.

6. The method according to claim 1, wherein said cell sample is a lymph node.

7. The method according to claim 1, wherein said cell sample is a tissue sample.

8. The method according to claim 1, wherein said human donor is suffering from an immunologic disorder.

9. The method according to claim 8, wherein said immunologic disorder is immunosuppression in a cancer patient.

10. The method according to claim 8, wherein said immunologic disorder is an autoimmune disease.

11. A population of cells comprising at least 80% human T regulatory cells, wherein said cells are obtained by the method comprising:
obtaining a cell sample comprising human T regulatory cells from a human donor;
contacting said cell sample with reagents that specifically recognize CD4, and CD25;
selecting for those cells that are CD4⁺CD25⁺, to provide an enriched population of regulatory T cells.

12. The population of cells according to claim 11, wherein said regulatory T cells are characterized as CD69⁻.

13. The population of cells according to claim 11, wherein said regulatory T cells are characterized as CD30⁺.

14. The population of cells according to claim 11, wherein said regulatory T cells are characterized as CCR6⁺.

15. The population of cells according to claim 11, wherein said cells are in a regulatory state.

16. The population of cells according to claim 11, wherein said cells are in a proliferative state.

17. An in vitro cell culture, comprising the enriched population of cells having regulatory T cell activity of claim 11.

18. The in vitro cell culture of claim 16, further comprising one or more subsets of human dendritic cells.

19. A method of assessing the immunologic state of a patient suffering from an immunologic disorder, the method comprising:
obtaining a cell sample suspected of comprising human T regulatory cells from said patient;
contacting said cell sample with reagents that specifically recognize CD4, and CD25;
identifying those cells that are CD4⁺CD25⁺,
determining one or more of: the absolute number, comparative number, tissue localization and antigenic specificity of CD4⁺CD25⁺ T regulatory cells in said patient.

20. The method according to claim 19, wherein said immunologic disorder is immunosuppression in a cancer patient.

21. The method according to claim 19, wherein said immunologic disorder is an autoimmune disease.

22. A method of modulating the trafficking of regulatory T cells in a human host, the method comprising:
administering an effective amount of a CCR6 modulating agent, in a dose effective to modulate said trafficking of regulatory T cells.

23. The method of claim 22, wherein said administration provides for a prolonged localized concentration of said CCR6 modulating agent.

24. The method of claim 22, wherein said CCR6 modulating agent is a CCR6 agonist.

25. The method of claim 24, wherein said CCR6 agonist is selected from the group consisting of LARC and MIP-3alpha.

26. A method of increasing the number of Treg cells in a mammal, the method comprising:
administering an effective dose of Flt3-L;
wherein the number of Treg cells is increased.
